(19) United States
(12) Patent Application Publication
(10) Pub. No.: US 2021/0304885 A1
(43) Pub. Date: Sep. 30, 2021

(54) METHOD AND DEVICE FOR CONTROLLING A MEDICAL APPARATUS

(71) Applicant: Siemens Healthcare GmbH, Erlangen (DE)

(72) Inventor: Thomas ALLMENDINGER, Forchheim (DE)

(73) Assignee: Siemens Healthcare GmbH, Erlangen (DE)

(21) Appl. No.: 17/202,634

(22) Filed: Mar. 16, 2021

(30) Foreign Application Priority Data
Mar. 25, 2020 (DE) 10 2020 203 848.1

(51) Int. Cl. G16H 40/40; G16H 50/30; G16H 50/20

(52) U.S. Cl. CPC G16H 40/40; G16H 50/20; G16H 50/30

(57) ABSTRACT

A method for controlling a medical apparatus. An embodiment of the method includes providing, via an interface, a first function dataset of a patient, measured within a first time interval; applying, via a processor, a trained function to the measured first function dataset provided, to estimate a second function dataset of the patient, predicted for a second time interval, wherein at least one parameter of the trained function is adapted based upon a comparison between a predicted, second training function-dataset for a second training time-interval, the second training function-dataset being predicted based upon a first training function-dataset of a training patient for a first training time-interval, and a comparison function-dataset of the training patient for the second training time-interval, and wherein the first training function-dataset and the comparison function-dataset are associated; and controlling, via a controller, the medical apparatus based upon the estimate.

### PRIORITY STATEMENT

The present application hereby claims priority under 35 U.S.C. §119 to German patent application number DE 102020203848.1 filed Mar. 25, 2020, the entire contents of which are hereby incorporated herein by reference.

### FIELD

Embodiments of the invention generally relate to a method and a device for controlling a medical apparatus, a training method and a training device for providing a trained function for use in a method for controlling a medical apparatus, and also to a medical apparatus, a computer program product and a computer-readable storage medium.

### BACKGROUND

For diagnosis or treatment of a patient, it can be provided that a medical apparatus used for the diagnosis or treatment of a patient is controlled on the basis of medical data of the patient. For example, it can be provided that control of an imaging sequence via a computed tomography apparatus (CT apparatus) is based on an EKG signal or a breath signal for the patient. This can include having to extrapolate an estimated future course of the measurement data on the basis of already measured measurement data in order to allow control of the system in a manner that is matched to the medical data.

A specific example, for instance, is acquiring CT data used to produce images of the heart of a patient. It is usual in this case to record the EKG of the patient and to use information derived therefrom to control the data acquisition, where the required system time instants for controlling the CT apparatus for the data acquisition can be determined on the basis of an estimated future course of the EKG.

As things currently stand, predictions for controlling medical apparatuses, for instance in predicting cardiac cycles, are often made by methods that involve finding a mean or median value over the measurement-data history, if applicable using simple enhancements by including linear trends or similar simple models.

### SUMMARY

The inventors have discovered that precise and well-matched control of the medical apparatus, however, can be crucial to high image quality, to avoiding artifacts in the data or to optimum treatment of a patient.

At least one embodiment of the present application provide an improved method and/or an improved device for controlling a medical apparatus.

The claims and the description below present further advantageous embodiments and developments of the invention, some of which are inventive in their own right.

At least one embodiment of the present invention relates to a method for controlling a medical apparatus. The method comprises the step of providing, via an interface, a first function dataset of a patient, which is measured within a first time interval. The method also comprises the step of applying, via a processing unit, a trained function to the provided, measured, first function dataset and thereby of estimating a second function dataset of the patient, which is predicted for a second time interval. At least one parameter of the trained function is adapted on the basis of a comparison of a predicted second training function-dataset for a second training time-interval, which is based on a first training function-dataset of a training patient for a first training time-interval, with a comparison function-dataset of the training patient for the second training time-interval. The first training function-dataset and the comparison function-dataset are associated with each other. In addition, the method comprises the step of controlling, via a control unit, the medical apparatus on the basis of the estimate. In other words, the medical apparatus is controlled on the basis of the predicted second function dataset of the patient.

According to an embodiment of the invention, the input data for the trained function can comprise the first, measured function dataset of the patient for the first time interval. According to an embodiment of the invention, the output data can comprise, in particular, the predicted second function dataset of the patient for the second time interval.

According to an embodiment of the invention, in the training phase of the trained function, a training function-dataset, preferably a plurality of training function-datasets, based on a first training time-interval of a training patient, preferably of a plurality of training patients, can be used in particular as the training input data. A predicted training function-dataset can be estimated on the basis thereof as the training output data. In addition, at least one parameter of the trained function can then be adapted on a comparison of the predicted training function-dataset of the training patient with a comparison function-dataset of the training patient as the training comparison data.

The control on the basis of the estimate obtained by applying the trained function can comprise controlling the medical apparatus or at least one component of the medical apparatus via the control unit. For example, a control signal that can be provided for the control unit for controlling the medical apparatus can be derived on the basis of the estimate. The controlling can comprise in particular controlling in a time interval that is in the future in relation to the first time interval. The controlling can comprise adjusting configuration parameters of the medical apparatus, or starting, stopping or adjusting a movement or state of the medical apparatus. The control can be initiated by the estimate, i.e. for example a specific defined procedure can be triggered on the basis of the estimate. The estimate can also be incorporated further into the control, and influence this control and hence the procedure of controlling the medical apparatus.

The control can be designed in the sense of real-time control. The measured first function dataset can then be referred to in particular as a real-time dataset, on the basis of which real-time control of the apparatus can be performed. The control of the apparatus is based here on the second function data for the second time interval, which second function data is estimated, i.e. predicted, on the basis of the first, measured function dataset.

A medical apparatus of an embodiment can comprise a medical apparatus in which control can be provided on the basis of patient data, in particular on the basis of patient data of the patient that is obtained in immediate relation in time to the control. For example, the medical apparatus may comprise a medical imaging apparatus. A specific example may be a computed tomography apparatus or a C-arm X-ray apparatus in which control can be provided on the basis of a measured EKG dataset or a measured respiratory graph of the patient. The medical apparatus can comprise a medical apparatus intended for treating the patient. This can comprise, for instance, a cancer treatment radiotherapy apparatus. The medical apparatus may also comprise an apparatus that dispenses the medication for a patient. In this case, the control can comprise timed control or control in terms of calculating a medication dose to be administered. A specific example may be providing a predicted function dataset for calculating control of an insulin pump on the basis of a blood sugar measurement of the patient. Control of other medical apparatuses apart from the examples specifically mentioned is also possible, however, within the scope of embodiments of the invention.

The method according to an embodiment of the invention advantageously allows reliable control of a medical apparatus on the basis of a predicative estimate of patient parameters. Controlling a medical apparatus on the basis of predicted data may be necessary in order to match the control of the medical apparatus, or of the procedure used on the patient, to currently existing patient parameters, and to ensure optimum control despite, for instance, a lead time being required for the control. Furthermore, an accurate prediction may be needed for ensuring complete or optimum data acquisition by the medical apparatus. Advantageously it can be made possible to match accurately to the patient or a patient parameter the required system time instants for controlling the medical apparatus, or to adjust settings of the medical apparatus sufficiently early for an optimum match to the patient or a specific situation of the patient. In particular, this can also help to perform medical applications on a patient efficiently in terms of dose and time. In particular, the method according to the invention advantageously allows a prediction that is both better and highly time-efficient even when there are complex relationships and dependencies in a patient parameter, so that delays in the procedure can be avoided.

An embodiment of the invention also relates to a training method for providing a trained function for use in a proposed method for controlling a medical apparatus. The training method comprises the steps of first providing, applying, adapting, and second providing.

An embodiment of the invention also relates to a device for controlling a medical apparatus comprising a processing unit having a computing unit and an interface. The device also comprises a control unit.

An embodiment of the invention also relates to a medical apparatus comprising a proposed device for controlling a medical apparatus.

An embodiment of the invention also relates to a training device for providing a trained function. The trained function can be provided in particular for use in a proposed method for controlling a medical apparatus. The training device advantageously comprises a training computing unit and a training interface.

An embodiment of the invention also relates to a computer program product comprising a computer program, which can be loaded directly into a memory of a processing unit, and which contains program segments in order to perform all the steps of the method for controlling a medical apparatus or of embodiments of the method when the program segments are executed by the processing unit.

An embodiment of the invention also relates to a computer program product comprising a computer program, which can be loaded directly into a training memory of a training device, and which contains program segments in order to perform all the steps of the method for providing a trained function or of one of the embodiments of the method when the program segments are executed by the training device.

An embodiment of the invention also relates to a computer-readable storage medium, on which are stored program segments which can be read and executed by a processing unit in order to perform all the steps of embodiments of the method for controlling a medical apparatus or of embodiments of the method when the program segments are executed by the processing unit.

An embodiment of the invention also relates to a computer-readable storage medium, on which are stored program segments which can be read and executed by a training device in order to perform all the steps of the method for providing a trained function or of one of the embodiments of the method when the program segments are executed by the training device.

In addition, an embodiment of the invention can also relate to a computer program or a computer-readable storage medium comprising a trained function provided by a method for providing a trained function or one of the embodiments of the method.

In addition, an embodiment of the invention can also relate to a method for controlling a medical apparatus, comprising:
providing, via an interface, a first function dataset of a patient, measured within a first time interval;
applying, via a processor, a trained function to the measured first function dataset provided, to estimate a second function dataset of the patient, predicted for a second time interval, wherein at least one parameter of the trained function is adapted based upon a comparison between a predicted, second training function-dataset for a second training time-interval, the second training function-dataset being predicted based upon a first training function-dataset of a training patient for a first training time-interval, and a comparison function-dataset of the training patient for the second training time-interval, and wherein the first training function-dataset and the comparison function-dataset are associated; and
controlling, via a controller, the medical apparatus based upon the estimate.

In addition, an embodiment of the invention can also relate to a training method for providing a trained function, comprising:
providing, via a training interface, a first training function-dataset of a training patient for a first training time-interval and an associated comparison function-dataset of the training patient for a second training time-interval;
applying, via a training computer, the trained function to the first training function-dataset provided to estimate a predicted, second training function-dataset of the training patient in the second training time-interval;
adapting, via the training computer, at least one parameter of the trained function based upon a comparison of the predicted, second training function-dataset and a corresponding comparison function-dataset in the second training time-interval; and
providing, via the training interface, the trained function.

In addition, an embodiment of the invention can also relate to a device for controlling a medical apparatus, comprising:
a processor including a computing device;
an interface designed to provide a first function dataset of a patient, measured within a first time interval; and
a computing unit, designed to apply a trained function to the first function dataset measured, for estimating a predicted, second function dataset of the patient in a second time interval, at least one parameter of the trained function being adapted based upon a comparison of a second training function-dataset for a second training time interval, the second training function-dataset being predicted based upon a first training function-dataset of a training patient for a first training time-interval, and a corresponding comparison function-dataset of the training patient for the second training time-interval; and
a controller, is designed to control the medical apparatus based upon the predicted, second function dataset of the patient, once estimated.

In addition, an embodiment of the invention can also relate to a training device for providing a trained function, comprising:
a training interface designed to provide a first training function-dataset of a training patient for a first training time-interval and an associated comparison function-dataset of the training patient for a second training time-interval; and
a training computing unit
   designed to apply a trained function to the first training function-dataset to estimate a predicted, second training function-dataset of the training patient in the second training time-interval,
   designed to adapt at least one parameter of the trained function based upon a comparison of the predicted, second training function-dataset and the corresponding comparison function-dataset in the second training time-interval, and
   designed to provide the trained function.

In addition, an embodiment of the invention can also relate to a non-transitory computer program product storing a computer program, directly loadable into a memory of a processor, including program segments to perform the method of an embodiment when the program segments are executed by the processor.

In addition, an embodiment of the invention can also relate to a non-transitory computer-readable storage medium, storing program segments, readable and executable by a processor, to perform the method of an embodiment when the program segments are executed by the processor.

In addition, an embodiment of the invention can also relate to a non-transitory computer program product storing a computer program, directly loadable into a training memory of a training device, including program segments to perform the method of an embodiment when the program segments are executed by the training device.

In addition, an embodiment of the invention can also relate to a non-transitory computer-readable storage medium, storing program segments, readable and executable by a training device, to perform the method of an embodiment when the program segments are executed by the training device.

### BRIEF DESCRIPTION OF THE DRAWINGS

The invention is described below using example embodiments with reference to the accompanying figures. Schematic, highly simplified diagrams that are not necessarily to scale appear in the figures, in which:

FIG. 1 is a schematic diagram of a method procedure of a method for controlling a medical apparatus;

FIG. 2 is a schematic diagram of an example data flow of a method procedure of a method for controlling a medical apparatus;

FIG. 3 is a timeline diagram illustrating the method procedure;

FIG. 4 is a schematic diagram of a method procedure of a training method for providing a trained function;

FIG. 5 is a schematic diagram of an example data flow of a method procedure of a training method for providing a trained function;

FIG. 6 is a schematic diagram of a device for controlling a medical apparatus, and of a training device for providing a trained function;

FIG. 7 is a schematic diagram of an example of a medical apparatus; and

FIG. 8 is a schematic diagram illustrating a case in which the method for controlling a medical apparatus is used.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated.The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the present invention relates to a method for controlling a medical apparatus. The method comprises the step of providing, via an interface, a first function dataset of a patient, which is measured within a first time interval. The method also comprises the step of applying, via a processing unit, a trained function to the provided, measured, first function dataset and thereby of estimating a second function dataset of the patient, which is predicted for a second time interval. At least one parameter of the trained function is adapted on the basis of a comparison of a predicted second training function-dataset for a second training time-interval, which is based on a first training function-dataset of a training patient for a first training time-interval, with a comparison function-dataset of the training patient for the second training time-interval. The first training function-dataset and the comparison function-dataset are associated with each other. In addition, the method comprises the step of controlling, via a control unit, the medical apparatus on the basis of the estimate. In other words, the medical apparatus is controlled on the basis of the predicted second function dataset of the patient.

The measured, first function dataset can comprise a measured dataset that has been obtained by a measurement on the patient or can comprise a parameter derived therefrom. The function dataset can comprise a parameter of the patient that represents a condition of a body function of the patient. The measured, first function dataset can comprise, or be derived from, a dataset relating to a vital-sign parameter of the patient. Such a parameter of a patient can relate to a basic function of the human body, in particular relating to breathing or circulation, for instance to the function of the heart. For example, the function dataset can be based on a dataset that comprises a heart rate, a blood pressure, a respiratory rate or an EKG dataset (electrocardiogram dataset). In further embodiment variants, the first, measured function dataset can also relate to another parameter of the patient, for instance to a blood sugar level or a medication level in the blood of the patient or such like.

The first, measured function dataset may be based, in particular, on a dataset that was measured within, i.e. during, the first time interval. The dataset comprised by the first, measured function dataset or from which the first, measured function dataset is derived can then comprise a time-resolved dataset within the first time interval. The first, measured function dataset can comprise, in particular, a plurality or sequence of values of a parameter of the patient, which parameter values are repeatedly measured directly within the first time interval or derived from a dataset measured in a time-resolved manner within the first time interval. The plurality of measured or derived values can represent a value progression, i.e. a history of the parameter under consideration of the patient, within the first time interval.

For example, the time length of a particular cardiac phase or of the entire cardiac cycle, for instance the time period between every two R-spikes (R-spike: highest spike in the electrocardiogram of a particular cardiac cycle of greatest electrical activity in the EKG dataset), can be derived from an EKG dataset measured within the first time interval. The function dataset can then comprise, for example, a plurality of values obtained for the time length of this cardiac phase or of the cardiac cycle for at least some, preferably for all, of the cardiac cycles comprised by the EKG dataset and recorded within the first time interval. A further example can comprise that a blood pressure of the patient is measured repeatedly within the first time interval. The function dataset can then comprise, for instance, a plurality of values for the blood pressure within the first time interval. The equivalent can also apply to other datasets or parameters of the patient.

Estimating the predicted, second function dataset of the patient for the second time interval can comprise estimating and thus predicting at least one predicted future value, preferably a plurality of predicted future values, or a further future course of the measured function dataset in the second time interval. Normally, future parameter values of the same parameter in the second time interval are predicted on the basis of the measured or derived parameter values comprised by the first function dataset.

The first time interval in this case may not necessarily be defined initially on the basis of a time unit, for instance seconds or minutes, and hence may not necessarily comprise a defined timespan or time length, for instance 20 seconds, 30 seconds, 1 minute or one day. The time length of the first time interval can be defined in particular by a requirement placed on the first, measured function dataset to have available a certain number of measured or derived values. For example, the requirement can include that the first function dataset comprises 5, 10, 20 or another number of measured or derived values of a parameter. The first time interval then extends over the time length in which the requirement-compliant number, for instance 5, 10 or 20, of values are measured or the measured dataset is sufficient for deriving the number of values. The requirement placed on the first function dataset may be, in particular, a requirement of the trained function applied to the function dataset. In particular, the second time interval, particularly the end time, may also not necessarily be defined initially on the basis of a time unit or by a time length or timespan, for instance a time length of 3 seconds, 10 seconds, 30 seconds or a number of minutes. The time length of the second time interval may be defined by a number of future values to be predicted by the trained function. For example, the trained function may be trained to estimate a certain number of values. The first and second time intervals may be predetermined in particular by the structure and design of the trained function used for the estimate.

For example, the measured first function dataset comprises the last 10 or 15 time periods between two R-spikes in an EKG dataset of the patient. The predicted second function dataset comprises, for example, a prediction for the 3 or 4 time periods between every two R-spikes, which time periods succeed the 10 measured time periods. The first time interval can then be determined by the timespan up to the 10 or 15 most recent time periods between two R-spikes. The second time interval can then be determined by the timespan in which the predicted 3 or 4 time periods between two R-spikes are expected. There may also be other configurations as well, however.

The first time interval can lie, in particular, in the time leading up to a planned control of the medical apparatus. In particular, it can be related directly in time to a subsequent control of the medical apparatus. The control of the medical apparatus is then based on the second function dataset, which is estimated, i.e. predicted, on the basis of the first, measured function dataset, for the second time interval.

In particular, the first time interval can preferably be longer than the second time interval. This means that preferably on the basis of a longer time history, a prediction is made that is shorter in time relative thereto. A relatively longer time history implies a more extensive measured function dataset compared with the extent of the predicted function dataset. This can advantageously ensure a more accurate estimate.

In particular, the length of the first time interval can be dependent on a timescale of an influence of influencing variables or influencing events on a patient parameter under consideration, or on a timescale of predictable regularly or irregularly occurring patterns in the first, measured function dataset. By selecting a suitable time interval, the prediction in the second time interval can thereby advantageously take into account these patterns and influences. In particular, the second time interval can be dependent on the timespans or lead times needed for the control.

Providing the first, measured function dataset can comprise retrieving from a measuring apparatus, or transferring therefrom to the processing unit, via the interface, a dataset of a patient, which measuring apparatus is designed to measure the dataset which the function dataset comprises or from which the function dataset is derived. For example, the measuring apparatus may be in the form of an EKG measuring apparatus, a blood pressure meter or a measuring apparatus for monitoring respiration of the patient. The providing can comprise that, via the interface, a measured function dataset, which is derived from a measured dataset, is provided after the function dataset has been derived for applying the trained function. The processing unit itself can be designed, for example, to derive the function dataset from a measured dataset of the patient, in which case then the function dataset can be provided via an interface for applying the trained function.

In particular, estimating the predicted, second function dataset comprises applying a trained function to the first, measured function data. An estimate of the predicted second function dataset via a trained function can advantageously allow an estimate that is both particularly accurate and time-efficient. In particular, this can ensure particularly advantageous results when it is necessary to take into account more complex relationships, for instance also including additional external sources of influence on the measured dataset of the patient that forms the basis of the first function dataset, or irregularly occurring patterns in the first function dataset.

A trained function can be implemented preferably via an artificial intelligence system, i.e. by a machine learning technique. An estimation based on applying a trained function can take better account of all relevant influencing variables, including those for which a user is unable to assess a connection to the estimation. An artificial intelligence system can refer to a system for artificially generating knowledge from experience. An artificial system learns from examples in a training phase, and can generalize once the training phase is finished. The use of such a system can comprise recognizing patterns and regularities in the training data. After the training phase, the artificial intelligence system can extract in hitherto unknown measurement data, for example, features or characteristics which are included in the estimation. After the training phase, the optimized, i.e. trained, algorithm can estimate a second function dataset on the basis of a hitherto unknown measured function dataset, for example. The artificial intelligence system may be based on an artificial neural network or on another machine learning technique. In particular, a trained function based on an artificial intelligence system can be used after the training phase to facilitate a prediction of the second function dataset in an automated and particularly reliable and time-efficient manner.

A trained function in particular maps input data onto output data. The output data may depend in particular also on one or more parameters of the trained function. The one or more parameters of the trained function can be determined and/or adapted by training. Determining and/or adapting the one or more parameters of the trained function can be based in particular on a pair composed of training input data and corresponding, i.e. associated, comparison output data, wherein the trained function for generating training output data is applied to the training input data. In particular, the determining and/or adapting can be based on a comparison of the training output data and the training comparison data. In general, a trainable function, i.e. a function containing one or more parameters yet to be adapted, is also referred to as a trained function.

Other terms for trained function are trained mapping rule, mapping rule containing trained parameters, function containing trained parameters, algorithm based on artificial intelligence, and machine-learning algorithm. An example of a trained function is an artificial neural network, where the edge weights of the artificial neural network are equivalent to the parameters of the trained function. The term “neural net” can also be used instead of the term “neural network”. In particular, a trained function may also be a deep artificial neural network (deep neural network). Another example of a trained function is a “support vector machine”, and in particular other machine-learning algorithms may also be used as the trained function.

The trained function can be trained in particular via backpropagation. First, training output data can be determined by applying the trained function to training input data. Thereafter, a difference between the training output data and the training comparison data can be ascertained by applying an error function to the training output data and the training comparison data. In addition, at least one parameter, in particular a weight, of the trained function, in particular of the neural network, can be adapted iteratively on the basis of a gradient of the error function with respect to the at least one parameter of the trained function. The difference between the training output data and the training comparison data can thereby advantageously be minimized during the training of the trained function.

The trained function, in particular the neural network, advantageously comprises an input layer and an output layer. The input layer can be designed to receive input data. In addition, the output layer can be designed to provide output data. The input layer and/or the output layer can each comprise a plurality of channels, in particular neurons.

According to an embodiment of the invention, the input data for the trained function can comprise the first, measured function dataset of the patient for the first time interval. According to an embodiment of the invention, the output data can comprise, in particular, the predicted second function dataset of the patient for the second time interval.

According to an embodiment of the invention, in the training phase of the trained function, a training function-dataset, preferably a plurality of training function-datasets, based on a first training time-interval of a training patient, preferably of a plurality of training patients, can be used in particular as the training input data. A predicted training function-dataset can be estimated on the basis thereof as the training output data. In addition, at least one parameter of the trained function can then be adapted on a comparison of the predicted training function-dataset of the training patient with a comparison function-dataset of the training patient as the training comparison data.

The control on the basis of the estimate obtained by applying the trained function can comprise controlling the medical apparatus or at least one component of the medical apparatus via the control unit. For example, a control signal that can be provided for the control unit for controlling the medical apparatus can be derived on the basis of the estimate. The controlling can comprise in particular controlling in a time interval that is in the future in relation to the first time interval. The controlling can comprise adjusting configuration parameters of the medical apparatus, or starting, stopping or adjusting a movement or state of the medical apparatus. The control can be initiated by the estimate, i.e. for example a specific defined procedure can be triggered on the basis of the estimate. The estimate can also be incorporated further into the control, and influence this control and hence the procedure of controlling the medical apparatus.

The control can be designed in the sense of real-time control. The measured first function dataset can then be referred to in particular as a real-time dataset, on the basis of which real-time control of the apparatus can be performed. The control of the apparatus is based here on the second function data for the second time interval, which second function data is estimated, i.e. predicted, on the basis of the first, measured function dataset.

A medical apparatus of an embodiment can comprise a medical apparatus in which control can be provided on the basis of patient data, in particular on the basis of patient data of the patient that is obtained in immediate relation in time to the control. For example, the medical apparatus may comprise a medical imaging apparatus. A specific example may be a computed tomography apparatus or a C-arm X-ray apparatus in which control can be provided on the basis of a measured EKG dataset or a measured respiratory graph of the patient. The medical apparatus can comprise a medical apparatus intended for treating the patient. This can comprise, for instance, a cancer treatment radiotherapy apparatus. The medical apparatus may also comprise an apparatus that dispenses the medication for a patient. In this case, the control can comprise timed control or control in terms of calculating a medication dose to be administered. A specific example may be providing a predicted function dataset for calculating control of an insulin pump on the basis of a blood sugar measurement of the patient. Control of other medical apparatuses apart from the examples specifically mentioned is also possible, however, within the scope of embodiments of the invention.

The method according to an embodiment of the invention advantageously allows reliable control of a medical apparatus on the basis of a predicative estimate of patient parameters. Controlling a medical apparatus on the basis of predicted data may be necessary in order to match the control of the medical apparatus, or of the procedure used on the patient, to currently existing patient parameters, and to ensure optimum control despite, for instance, a lead time being required for the control. Furthermore, an accurate prediction may be needed for ensuring complete or optimum data acquisition by the medical apparatus. Advantageously it can be made possible to match accurately to the patient or a patient parameter the required system time instants for controlling the medical apparatus, or to adjust settings of the medical apparatus sufficiently early for an optimum match to the patient or a specific situation of the patient. In particular, this can also help to perform medical applications on a patient efficiently in terms of dose and time. In particular, the method according to the invention advantageously allows a prediction that is both better and highly time-efficient even when there are complex relationships and dependencies in a patient parameter, so that delays in the procedure can be avoided.

According to an embodiment variant of the method according to the invention, the method can comprise that the first time interval and the measured, first function dataset for the first time interval are updated, and a predicted, second function dataset is re-estimated for an updated, second time interval by applying the trained function to the updated, measured first function dataset.

This variant can comprise that, over a timespan that is longer than the time length of the first time interval, second function datasets are predicted continuously on the basis of respective updated, first measured function datasets in respective updated, first time intervals. The control can then be based, for instance, on the most recent prediction for the second function dataset of the patient. The control can comprise adjusting the control on the basis of the most recent prediction for the second function dataset. A rolling prediction can thereby be provided, in which a rolling adjustment of the first time interval takes place and each new prediction takes into account the data obtained between two respective predictions. Thus accurate predictions for the second time interval can advantageously always be guaranteed to be continuously available over a prolonged timespan. Advantageously, an up-to-date prediction can always be provided for the control.

The method for controlling a medical apparatus and aspects of the method can then be applied in this case equally without restrictions likewise to the updated, measured, first function dataset and to the predicted, second function dataset for an updated, second time interval. This means that when referring below to a first or second function dataset or first or second time interval, then this can apply equally to an updated first or second function dataset respectively or an updated first or second function dataset respectively.

According to an embodiment variant of the method, the second time interval is adjacent in time to the first time interval.

The estimate of a function dataset for an immediately adjacent time interval advantageously allows, in particular, real-time control of the medical apparatus on the basis of currently existing data for the patient. At the same time, it is possible to minimize a control time-delay. In addition, when timespans are immediately adjacent, it is advantageously possible to minimize differences between the prediction and an actual course of the function dataset of the patient, and hence to ensure accurate control.

If the second time interval for which the function dataset is predicted is immediately adjacent to the first time interval, this can lead in particular to a requirement for a prediction that is as instantaneous as possible. This means that the shortest possible time is provided for the prediction. In particular, the time required for the prediction can be limited to a, preferably smallest possible, fraction of the second time interval, for instance a fraction in the region of 0.1% or less. The required time length within which the second, predicted function dataset is estimated via the trained function preferably comprises less than 200 ms, more preferably less than 50 ms, for example 30 ms or 20 ms. This can advantageously ensure that the predicted second function dataset is available, and provided for control on the basis thereof, as soon as possible. This can also allow the medical apparatus to be controlled already at an initial stage or during the course of the second time interval. Time delays can advantageously be minimized. In particular, applying a trained function can allow a time-efficient prediction here without delays necessary even in the case of complex relationships.

According to an embodiment variant of the method according to the invention, the medical apparatus is then controlled within the second time interval and/or in a third time interval immediately adjacent to the second time interval. The method according to the invention advantageously allows control with a minimum time delay.

The trained function can preferably comprise a neural network. For example, the trained function can comprise a feed-forward neural network, a recurrent neural network, or a convolutional neural network (CNN) or a network comprising a convolutional layer.

The inventors have discovered that implementing the trained function in the form of a neural network can ensure particularly advantageously an estimate that is both accurate and time-efficient.

In a preferred variant, the trained function in particular comprises a feed-forward network. A feed-forward network consists of an input layer containing the input values, i.e. the input data, one or more hidden layers consisting of a plurality of neurons, and an output layer consisting of one or more neurons, on the basis of which the output values are output. In pure feed-forward networks, the neuron outputs are passed only in the direction of processing and not fed back through a recurrent edge. In other words, forward propagation is used to calculate the output of a feed-forward neural network. The outputs of the second layer are calculated on the basis of the input data. Then the outputs of the third layer are calculated on the basis of the outputs from the neurons in the second layer, and so on. Generally there is no feedback from neurons nor are there connections between neurons within a layer or connections across layers. In particular, back-propagation can then be used to train, i.e. adapt, the weights of the connections of a feed-forward neural network on the basis of the training data.

A neuron of a feed-forward network can essentially calculate a linear combination of the inputs to the neuron, weighted by the weight of the particular connection between the respective neurons, and apply an activation function g: R→[0,1] to the result in order to determine the output from the neuron. An activation function in a feed-forward network may be based, for example, on a logistic function or on a hyperbolic tangent or on another function.

The number of degrees of freedom and the complexity can be minimized in the case of a feed-forward network. This can be particularly advantageous in a real-time application where there is also limited availability of time resources, and, if applicable, physical resources.

Other neural networks, however, can also be used apart from a feed-forward network. Thus in another advantageous embodiment variant, a recurrent neural network, for instance a long short-term memory network (LSTM), can be used for example. Recurrent neural networks contain cycles, i.e. a layer can receive as an input, output from a downstream layer.

According to an embodiment of the method according to the invention, the measured, first function dataset can be based on an EKG dataset of the patient, on a respiratory dataset of the patient, on a blood-pressure dataset of the patient, or on a blood-level dataset of the patient.

For example, the first function dataset can relate to a parameter, for instance a time length or time instant, of a cardiac phase or of a cardiac cycle of the patient. The first function dataset can relate to a respiratory parameter, for instance the time length or time instant of a respiratory phase. The first function dataset can comprise a blood-pressure parameter, for instance a blood-pressure level. The first function dataset can comprise, for instance, also a concentration of a medication in the blood of a patient or a blood sugar level. The predicted second function dataset can then comprise the prediction for the dataset or a parameter derived therefrom for the second time interval. If the first function dataset comprises, for example, a sequence of values for time lengths of the cardiac cycles of the patient within the first time interval, the predicted second function dataset can comprise one or more future time lengths of the cardiac cycles of the patient in the second time interval.

An implementation of the method on the basis of an EKG dataset of the patient, a respiratory dataset of the patient, a blood-pressure dataset or a blood-level dataset of the patient thus constitutes a particularly advantageous embodiment variant. These datasets are often incorporated in a control of a medical apparatus and can have a severe impact on the quality of the results achieved using the medical apparatus if the matching of the control is sub-optimum. In addition, in other embodiment variants, the measured first function dataset can also be based on another dataset of the patient.

According to a further embodiment variant of the method for controlling a medical apparatus, the measured, first function dataset of the patient is also influenced by an influencing event occurring during the first time interval. In this variant, the trained function is also adapted on the basis of a first training function-dataset of a training patient, which training function-dataset is influenced by a training influencing event.

An external influencing event may cause a, sometimes complex, response in the measured dataset of a patient that is comprised by the first function dataset or from which the first function dataset is derived. Moreover, such a response may follow a patient-specific course for different patients despite similarities in a theoretical course, and can be modeled only with difficulty using simple prediction models. An external influencing event can accordingly have a severe impact on a prediction, or can result in the need to delay control if it is necessary to wait for the measured patient parameters, to which the control is meant to be matched, to stabilize after the impact of the influencing event. Such a waiting period, however, can in turn have an adverse effect on the usage procedure, for instance a prolonged breath-holding phase for the patient or an acquisition sequence at a sub-optimum time relative to administering a contrast agent.

Even when an influencing event has an impact, the method according to an embodiment of the invention can advantageously facilitate an accurate estimate and in particular avoid the need for a time delay due to a waiting period after the influencing event. In particular, applying a trained function that is trained on the basis of training function-dataset(s) influenced by a training influencing event advantageously allows the estimate of the predicted, second function dataset to take account thereof. As a rule, this cannot be modeled, or only to a very limited extent, using conventional prediction models.

According to a preferred embodiment variant of the control method, the influencing event comprises at least one event from the following list:
a breath signal defined for the patient;
a medication for a patient; or
a contrast-agent injection.

A combination of influencing events may also be present. An event, comprising a breath signal, a medication or a contrast-agent injection, can occur particularly frequently in the lead-up to control of a medical apparatus, and can cause a response, which is usually patient-specific. This can hence lead to uncertainties in a prediction and in control of the medical apparatus based thereon. It is only possible with great difficulty to take into account such an event and its influence on a specific patient in a prediction based on conventional models. The method according to the invention comprising applying the trained function can advantageously allow an estimate even in these cases, because more complex relationships and patterns can also be recognized in the training data and applied to a specific patient. The inventors have discovered that in these cases of the influence on the patient parameters, the method according to the invention can be used particularly advantageously for an improved estimate, and, equally advantageously, it is possible to dispense with waiting for stabilization, and hence to allow an instantaneous prediction and thus also control.

In an embodiment variant of the method, the step of controlling comprises controlling a movement of the medical apparatus or of a component of the medical apparatus.

The controlling can comprise starting or stopping or adjusting, for instance accelerating, a movement of the medical apparatus or of moving components of the medical apparatus. For instance, a patient positioning device that is comprised by a medical apparatus and on which the patient is supported can be moved. This can include moving the patient positioning device to a specified position. This can be provided, for example, when the positioning of the patient is provided relative to a data acquisition unit, for instance to an X-ray source/X-ray detector combination, or relative to a treatment unit, for instance a gamma-radiation source. Controlling the movement can comprise accelerating the patient positioning device to a specified speed, or adjusting a speed of the patient positioning device. This can be provided, for example, when continuous movement and, if applicable, coordination of the movement of the patient on the patient positioning device, is provided for an image-data acquisition as part of a computed tomography acquisition or MR acquisition. The control can also relate to other components. For instance, the control can comprise a movement of a radiation source comprised by a medical apparatus relative to a patient. For example, this can comprise positioning the radiation source relative to a patient or controlling a rotational movement of the radiation source about a patient, for instance as part of a CT image acquisition sequence.

Alternatively or additionally, the method can include that the step of controlling comprises starting or stopping a data acquisition sequence of a data acquisition unit comprised by the medical apparatus. The data acquisition unit can comprise an X-ray detector, for example. The data acquisition unit can comprise an X-ray source/X-ray detector combination. The data acquisition unit can also comprise, for example, a coil arrangement in an MR apparatus or alternative data acquisition units. The data acquisition can advantageously be started or stopped at a suitable time instant.

In addition, the control can comprise activating or deactivating the medical apparatus or a component of the medical apparatus.

The control can also comprise controlling a computing unit comprised by the medical apparatus. For example, the control can comprise controlling a computing unit of the medical apparatus for calculating or adjusting the dispensing of medication.

The medical apparatus can be designed to emit X-ray and/or gamma radiation. Alternatively or additionally, the method can then include that the step of controlling comprises adjusting a radiation status of the medical apparatus. The step of controlling can comprise in particular adjusting a radiation status of an X-ray or gamma-ray source comprised by the medical apparatus. Adjusting the radiation status can comprise switching the radiation on or off or a dose modulation, for instance modulating the tube current of an X-ray tube that is emitting the X-ray radiation.

Advantageously, dose-efficient control that is matched to the patient, or optimum results obtained by the medical apparatus, can be guaranteed by matching the radiation, the data acquisition and/or a movement to the patient. Automated control and an automated usage procedure for using the medical apparatus on the patient are advantageously facilitated on the basis of the estimate.

An embodiment of the invention also relates to a training method for providing a trained function for use in a proposed method for controlling a medical apparatus. The training method comprises the steps of first providing, applying, adapting, and second providing.

The step of first providing comprises providing, via a training interface, a first training function-dataset of a training patient for a first training time-interval and providing an associated comparison function-dataset of the training patient for a second training time-interval. The step of applying comprises applying, via a training computing unit, the trained function to the provided first training function-dataset and thereby estimating a predicted, second training function-dataset of the training patient in the second training time-interval. The step of adapting comprises adapting, via the training computing unit, at least one parameter of the trained function on the basis of a comparison of the predicted, second training function-dataset and the corresponding comparison function-dataset in the second training time-interval. The step of second providing comprises providing, via the training interface, the trained function.

A trained function can advantageously be provided that allows an accurate and time-efficient estimation of a predicted function dataset of a patient.

Via a training interface are preferably provided a multiplicity of first training function-datasets of a multiplicity of training patients and of corresponding, i.e. associated, comparison function-datasets of the respective training patients, which function-datasets are incorporated into the training method and can form the basis for adapting the trained function.

The first training function-dataset and the comparison function-dataset can be based in particular on a shared measured dataset of the same training patient, which dataset can be separated for the training into the first training function-dataset and the comparison function-dataset. The shared dataset can comprise, for example, measurement data from the patient over a timespan comprising the first training time-interval and the second training time-interval. In general, however, artificially generated, i.e. simulated, training datasets are also conceivable.

The training function-dataset in particular can have all the attributes of the first, measured function dataset that were described with reference to the method for controlling a medical apparatus, and vice versa. The training function-dataset and the associated comparison function-dataset are preferably based on domain-specific measured function datasets from a real patient population of the medical apparatus or of the same apparatus group of the medical apparatus. This means that the function dataset used for the training method, and the function dataset on the basis of which an estimate is made or which is meant to be estimated, were/are determined preferably under the same conditions. For example, if control of a computed tomography apparatus is meant to be facilitated on the basis of an EKG dataset, in which the patients are also subject to a breath signal, a training function-dataset that is likewise based on EKG data of a training patient influenced by a breath signal is preferably used for the training method. In general, however, artificially generated, i.e. simulated, training datasets are also conceivable.

An embodiment of the invention also relates to a device for controlling a medical apparatus comprising a processing unit having a computing unit and an interface. The device also comprises a control unit.

The interface is designed to provide a first function dataset of a patient, which is measured within a first time interval. The computing unit is designed to apply a trained function to the measured, first function dataset for an estimate of a predicted, second function dataset of the patient in a second time interval. At least one parameter of the trained function is adapted on the basis of a comparison of a second training function-dataset for a second training time-interval, which second training function-dataset is predicted on the basis of a first training function-dataset of a training patient for a first training time-interval, and of a corresponding comparison function-dataset of the training patient for the second training time-interval. The control unit is designed to control the medical apparatus on the basis of the estimate.

The computing unit or the control unit can be designed to derive, on the basis of the estimate, a control signal for controlling the medical apparatus. In addition, the interface can be designed to output the estimate or a derived control signal for the control unit.

In particular, the device can be designed to control the medical apparatus in an automated manner on the basis of the estimate.

Such a device for controlling a medical apparatus can be designed in particular to perform the above-described methods according to the invention for controlling a medical apparatus, and aspects of the methods. The device can be designed to perform these methods and aspects thereof by virtue of the processing unit, which comprises the computing unit and the interface, and the control unit being designed to perform the relevant method steps.

The advantages of the proposed device are essentially the same as the advantages of the proposed method for controlling a medical apparatus. Features, advantages or alternative embodiments mentioned in this connection can also be applied to the device for the control, and vice versa.

The device is preferably designed for a real-time prediction of the second function dataset on the basis of the provided first, measured function dataset and for real-time control on the basis of the estimate.

An embodiment of the invention also relates to a medical apparatus comprising a proposed device for controlling a medical apparatus.

The medical imaging apparatus is advantageously designed to implement an embodiment of the proposed method for controlling the medical apparatus. The advantages of the proposed medical apparatus are essentially the same as the advantages of the proposed method for controlling a medical apparatus. Features, advantages or alternative embodiments mentioned in this connection can also be applied to the medical apparatus, and vice versa.

A medical apparatus may comprise a medical imaging apparatus. A medical imaging apparatus can be designed to acquire a two-dimensional or three-dimensional image dataset from the patient or from a region under examination of a patient. For example, the medical imaging apparatus may be in the form of a medical X-ray apparatus comprising an X-ray source and an X-ray detector opposite thereto, with the patient positioned for the image data acquisition between the X-ray detector and the X-ray source. The medical apparatus can be in the form of a computed tomography apparatus. The medical apparatus, however, may also be in the form of a C-arm X-ray apparatus and/or DynaCT, and/or a magnetic resonance system (MRI) and/or ultrasound apparatus, for example. The medical apparatus may comprise, however, for example also a radiotherapy apparatus containing a γ-radiation source. An apparatus of this type can be used, for instance, in cancer treatment for irradiating a tumor region in the patient. The medical apparatus may also comprise a medical apparatus for dispensing medication, for instance an insulin pump or the like.

In particular, the medical apparatus can be controlled on the basis of the predicted, second function dataset. The medical apparatus can advantageously be controlled in an automated manner on the basis of the predicted, second function dataset.

An embodiment of the invention also relates to a training device for providing a trained function. The trained function can be provided in particular for use in a proposed method for controlling a medical apparatus. The training device advantageously comprises a training computing unit and a training interface.

The training interface is designed to provide a first training function-dataset of a training patient for a first training time-interval and to provide an associated comparison function-dataset of the training patient for a second training time-interval. In addition, the training computing unit is designed to apply the trained function to the first training function-dataset for an estimate of a second, predicted training function-dataset of the training patient in the second training time-interval. The training computing unit is also designed to adapt at least one parameter of the trained function on the basis of a comparison of the predicted, second training function-dataset and the corresponding comparison function-dataset in the second training time-interval.

In addition, the training interface is designed to provide the trained function.

Such a training device can be designed in particular to perform the above-described methods according to the invention for providing a trained function, and aspects of the methods. The training device is designed to perform these methods and aspects thereof by virtue of the training interface and the training computing unit being designed to perform the relevant method steps.

An embodiment of the invention also relates to a computer program product comprising a computer program, which can be loaded directly into a memory of a processing unit, and which contains program segments in order to perform all the steps of the method for controlling a medical apparatus or of embodiments of the method when the program segments are executed by the processing unit.

An embodiment of the invention also relates to a computer program product comprising a computer program, which can be loaded directly into a training memory of a training device, and which contains program segments in order to perform all the steps of the method for providing a trained function or of one of the embodiments of the method when the program segments are executed by the training device.

An embodiment of the invention also relates to a computer-readable storage medium, on which are stored program segments which can be read and executed by a processing unit in order to perform all the steps of embodiments of the method for controlling a medical apparatus or of embodiments of the method when the program segments are executed by the processing unit.

An embodiment of the invention also relates to a computer-readable storage medium, on which are stored program segments which can be read and executed by a training device in order to perform all the steps of the method for providing a trained function or of one of the embodiments of the method when the program segments are executed by the training device.

In addition, an embodiment of the invention can also relate to a computer program or a computer-readable storage medium comprising a trained function provided by a method for providing a trained function or one of the embodiments of the method.

An implementation largely in software has the advantage that even processing units and/or training units already in use can be easily upgraded by a software update in order to work in the manner according to the invention. Such a computer program product may comprise in addition to the computer program, if applicable, extra elements such as e.g. documentation and/or extra components, and also hardware components such as e.g. hardware keys (dongles etc.) for using the software.

In the context of the invention, features described with regard to different embodiments of the invention and/or different claim categories (method, use, device, system, arrangement, etc.) can also be combined to give further embodiments of the invention. For example, a claim relating to a device can also be developed by combining with features described or claimed in connection with a method, and vice versa. Functional features of a method can be implemented by correspondingly designed object-related components. In addition to the embodiments of the invention described explicitly in this application, a person skilled in the art will be able to arrive at various further conceivable embodiments of the invention without departing from the scope of the invention defined by the claims.

The use of the indefinite article “a” or “an” does not rule out the possibility of there also being more than one of the feature concerned. The use of the expression “comprise” does not exclude the possibility of the terms linked by the expression “comprise” being identical. For example, the medical imaging device comprises the medical imaging device. The use of the expression “unit” does not exclude the possibility that the subject to which the expression “unit” relates comprises a plurality of components that are spatially separate from one another.

In the context of the present application, the expressions “on the basis of” and “based on” can be understood in particular in the sense of the expression “using”. In particular, any wording, according to which a first feature is produced (or obtained, defined etc.) on the basis of a second feature, does not exclude the possibility that the first feature is produced (or obtained, defined etc.) on the basis of a third feature.

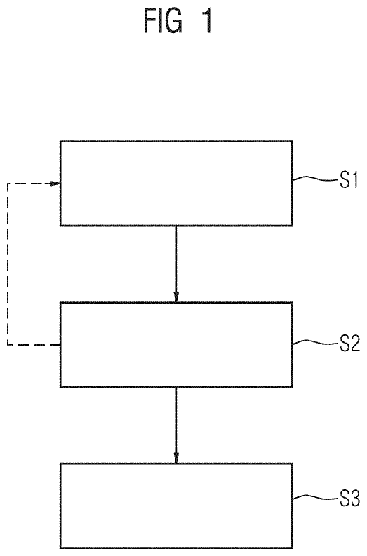

FIG. 1 shows a schematic diagram of a method procedure of a method for controlling a medical apparatus. The method comprises the step of providing S1, via an interface IF, first function dataset FD of a patient 39, which is measured within a first time interval dtm. The method also comprises the step of applying S2, via a processing unit 45, a trained function TF to the provided, measured, first function dataset FD and thereby of estimating a second function dataset EFD of the patient 39, which is predicted for a second time interval dtₑ. In this case, at least one parameter of the trained function TF is adapted on the basis of a comparison between a predicted second training function-dataset T-EFD for a second training time-interval, which is based on a first training function-dataset T-FD of a training patient for a first training time-interval, and a comparison function-dataset T-VFD of the training patient for the second training time-interval, wherein the first training function-dataset T-FD and the comparison function-dataset are associated with each other. In addition, the method comprises controlling S3, via a control unit 51, the medical apparatus 32 on the basis of the estimate.

The trained function can be implemented preferably via an artificial intelligence system, i.e. by a machine learning technique. The trained function TF preferably comprises a neural network, for instance a feed-forward network.

The method can comprise (indicated by the dashed arrow in FIG. 1) that the first time interval dtm and the measured, first function dataset FD are updated, and a predicted, second function dataset EFD2 is re-estimated for an updated, second time interval dt_{e,2} by applying the trained function TF to the updated, measured, first function dataset FD₂.

The method for controlling a medical apparatus and aspects of the method can then be applied in this case equally without restrictions likewise to the updated, measured, first function dataset FD2 and to the predicted, second function dataset EFD2 for an updated, second time interval dt_{e,2}.

According to an embodiment variant, the measured, first function dataset FD can be based in particular on an EKG dataset of the patient 39, on a respiratory dataset of the patient 39, on a blood-pressure dataset of the patient 39, or on a blood-level dataset of the patient 39.

According to an embodiment variant, the step of controlling S3 can comprise in particular activating the medical apparatus 32 or a component 37,36,41 of the medical apparatus 32, controlling a movement of the medical apparatus 32 or of a component 37,36,41 of the medical apparatus 32, starting or stopping a data acquisition sequence via a data acquisition unit 36,37 comprised by the medical apparatus 32, or adjusting a radiation status of an X-ray source 37 or gamma-ray source comprised by the medical apparatus 32. The controlling can also comprise controlling a computing unit for the purpose of calculation, for instance calculating an adjusted medication dose or the like.

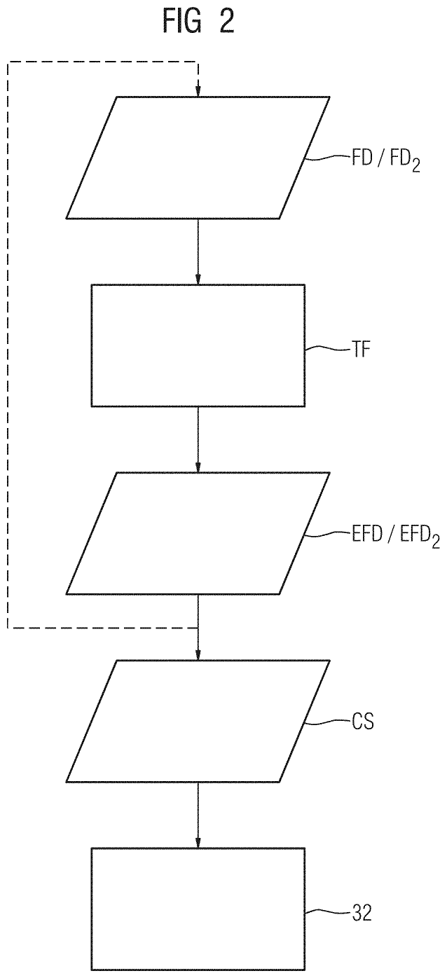

FIG. 2 shows a schematic diagram of an example data flow of a method procedure of a method for controlling a medical apparatus.

The first measured function data FD of the patient or updated first function data FD₂ of the patient is used as the input data for the trained function FT. Predicted second function data EFD or updated, second function data EFD₂ can be obtained by applying the trained function FT. On the basis of the predicted second function data, a control signal CS is derived for the control unit 51, which controls the medical apparatus 32 on the basis thereof.

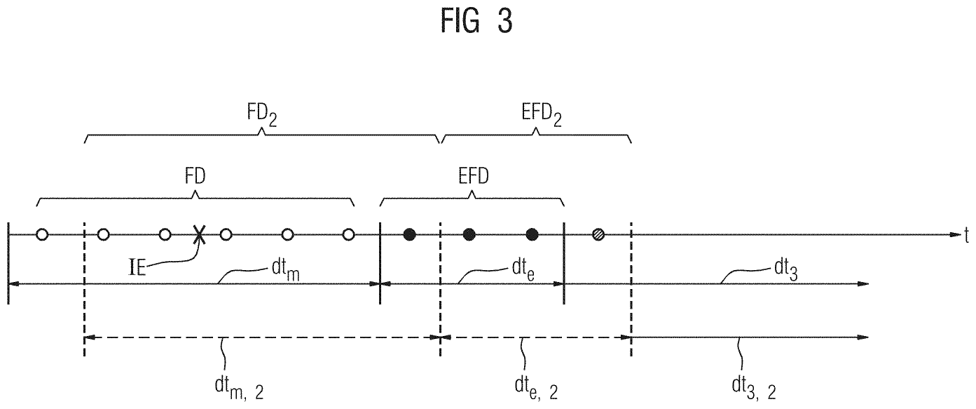

FIG. 3 shows a timeline diagram illustrating an example of a method procedure.

The first, measured function dataset FD is here illustrated as a sequence of a number of parameter values, measured within the first time interval dtₘ, in the form of circles plotted on the timeline. The parameter values can each represent a specific time instant or a time length of a cardiac phase or of a respiratory phase, or a measured course of another parameter value, for instance a repeatedly measured blood-pressure value. A predicted second function dataset EFD is estimated for the second time interval dtₑ on the basis of the first, measured function dataset FD. In the example shown, this predicted second function dataset EFD comprises the three future parameter values of the parameter under consideration in the second time interval dtₑ that follow the parameter values of the first function dataset FD.

According to an advantageous embodiment variant of the method, the second time interval dtₑ is adjacent, preferably immediately adjacent, in time to the first time interval dtm.

In addition, this illustration also shows an updated first time interval dt_{m,2}, so that by applying the trained function TF to the updated, measured, first function dataset FD₂ within the updated time interval dt_{m,2}, a predicted, second function dataset EFD₂ for an updated, second time interval dt_{e,2} is estimated and can be provided for a control. A rolling prediction can thereby be provided, in which a rolling adjustment of the first time interval takes place and each new prediction takes into account the data obtained between two respective predictions, in this case a first measured value that follows the original measurement interval dtₘ.

The medical apparatus can then be controlled in particular during the second time interval dtₑ and dt_{e,2} respectively and/or also in the third time interval dt₃ and dt_{3,2} respectively that adjoins the second time interval dtₑ and dt_{e,2} respectively.

In addition, the measured first function dataset FD of the patient 39 can also be influenced at least by an influencing event IE occurring during the first time interval dtm. In this case, the trained function TF is adapted likewise on the basis of a first training function-dataset T-FD of a training patient, which training function-dataset T-FD is influenced by a training influencing event.

An influencing event IE of this type can comprise a breath signal defined for the patient 39, a medication for the patient, or a contrast-agent injection. An external influencing event IE may cause a, sometimes complex, response in the measured dataset of a patient 39 that is comprised by the first function dataset or from which the first function dataset is derived. The prediction quality, i.e. the accuracy of the prediction, can hence be severely impaired by an external influencing event. Even when an external influencing event IE has an impact, the method according to the invention can advantageously facilitate an accurate estimate and in particular can help to avoid the need for a time delay due to a period waiting for the patient parameter to stabilize after the influencing event. This can allow instantaneous control.

The first time interval may not necessarily be defined initially on the basis of a time unit, and hence may not necessarily comprise a defined timespan or time length. The time length of the first time interval can be defined in particular by a requirement placed on the first, measured function dataset to have available a certain number of measured or derived values. In particular, the second time interval, particularly the end time, may also not necessarily be defined initially on the basis of a time unit or by a time length or timespan. The time length of the second time interval may be defined by a number of future values to be predicted by the trained function. The first and second time intervals may thus be predetermined in particular by the structure and design of the applied trained function, or may be predetermined by a user by the architecture of the trained function provided for the method.

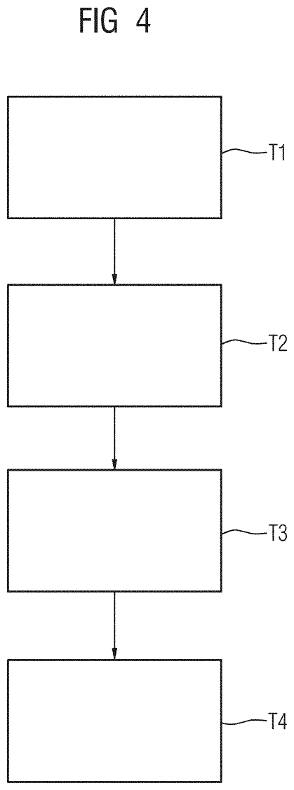

FIG. 4 shows a schematic diagram of a method procedure of a training method for providing a trained function. The trained function can be applied in an above-described method for controlling a medical apparatus.

The training method comprises the step of providing T1, via a training interface T-IF, a first training function-dataset T-FD of a training patient for a first training time-interval and an associated comparison function-dataset T-VFD of the training patient for a second training time-interval. The training method also comprises applying T2, via a training computing unit T-CU, the trained function TF to the provided first training function-dataset T-FD. A predicted, second training function-dataset T-EFD of the training patient in the second training time-interval is thereby estimated. The training method additionally comprises adapting T3, via the training computing unit T-CU, at least one parameter of the trained function TF on the basis of a comparison of the predicted, second training function-dataset T-FD and the corresponding comparison function-dataset T-VD in the second training time-interval. In addition, the training method comprises providing T4, via the training interface T-IF, the trained function TF.

The training function-dataset can preferably have all the attributes of the first, measured function dataset that were described with reference to the method for controlling a medical apparatus, and vice versa.

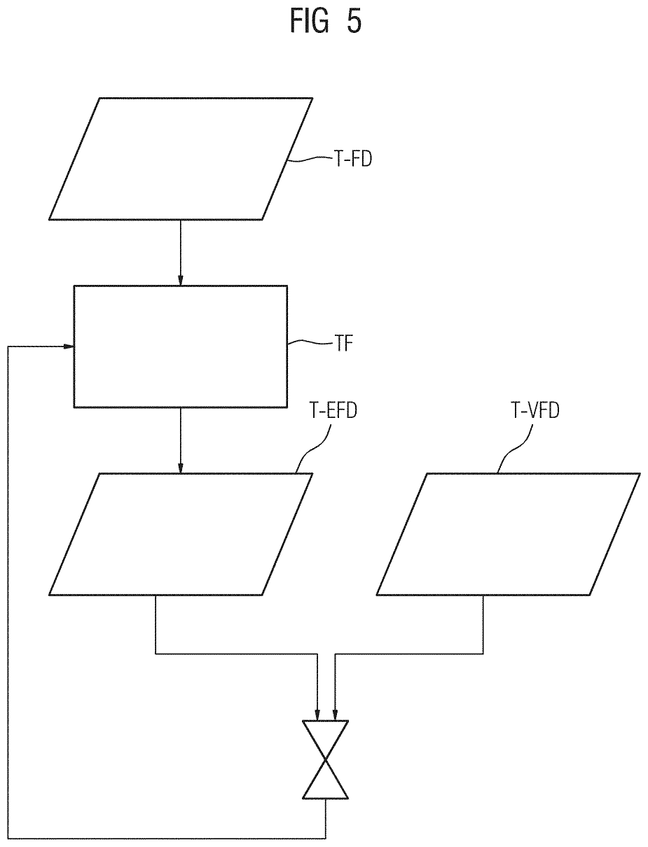

FIG. 5 shows a schematic diagram of an example data flow of a method procedure of a training method for providing a trained function.

A trained function TF is applied to the provided training function-dataset T-FD of the training patient for a first training time-interval in order to estimate a predicted second training function-dataset T-EFD. At least one parameter of the trained function TF is adapted on the basis of a comparison between the comparison function-dataset T-VFD of the training patient for a second training time-interval, which comparison function-dataset T-VFD is associated with the provided training function-dataset T-FD, and the predicted, second training function-dataset T-EFD.

This is preferably carried out using a plurality of provided training function-datasets T-FD of a plurality of training patients for a first training time-interval and associated comparison function-datasets T-VFD of a respective training patient for a second training time-interval.

The trained function can then be provided.

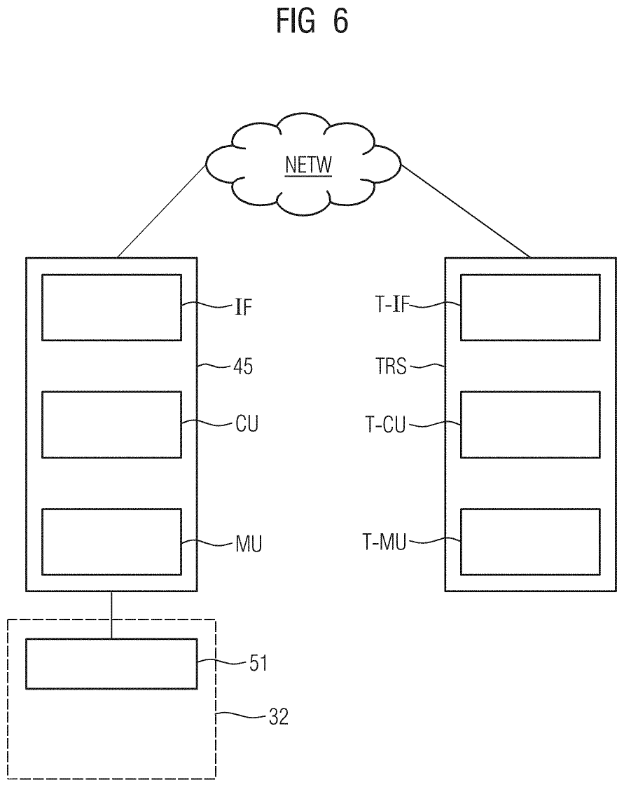

FIG. 6 shows a device for controlling a medical apparatus and a training device TRS for providing a trained function. The device comprising a processing unit 45 and a control unit 51 can advantageously be designed to perform a method according to the invention for controlling a medical apparatus. The training unit TRS shown can advantageously be designed to perform a proposed method for providing a trained function TF. The processing unit 45 can advantageously comprise an interface IF, a computing unit CU and a memory unit MU. In addition, the training unit TRS can advantageously comprise a training interface T-IF, a training computing unit T-CU and a training memory unit T-MU.

The interface IF can be designed to provide a first function dataset FD of a patient 39 that is measured within a first time interval dtm.

In addition, the computing unit CU can be designed to apply a trained function TF to a measured, first function dataset FD for an estimate of a predicted, second function dataset EFD of the patient 39 in a second time interval dtₑ, wherein at least one parameter of the trained function TF is adapted on the basis of a comparison of a second training function-dataset T-EFD for a second training time interval, which second training function-dataset T-EFD is predicted on the basis of a first training function-dataset T-FD of a training patient for a first training time-interval, and a corresponding comparison function-dataset T-VFD of the training patient for the second training time-interval. In addition, the computing unit CU can also be designed to derive a control signal CS for controlling the medical apparatus 32 on the basis of the estimate.

The interface IF can also be designed to provide the control signal CS for the control unit 51.

The control unit 51 can then be designed to control the medical apparatus 32 on the basis of a provided derived control signal CS.

The device comprising the processing unit 45 and the control unit 51 can be designed to perform a proposed method for controlling a medical apparatus 32. The proposed device for controlling a medical apparatus can be designed to perform the embodiment variants of the proposed method for controlling a medical apparatus 32 by virtue of the processing unit 45, which comprises the interface IF and the computing unit CU, and the control unit 52 being designed to perform the respective steps of the method.

The training interface T-IF can be designed to provide a first training function-dataset T-FD of a training patient for a first training time-interval and an associated comparison function-dataset T-VFD of the training patient for a second training time-interval.

The training computing unit T-CU can be designed to apply a trained function TF to the first training function-dataset T-FD for an estimate of the predicted, second training function-dataset T-EFD of the training patient in the second training time-interval. In addition, the training computing unit T-CU can be designed to adapt at least one parameter of the trained function TF on the basis of a comparison of the predicted, second training function-dataset T-EFD and the corresponding comparison function-dataset T-VFD in the second training time-interval. The second training function-dataset T-EFD corresponds to the comparison function-dataset T-VFD if the predicted, second training function-dataset T-EFD is based on a first training function-dataset T-FD that is associated with the comparison function-dataset.

In addition, the training interface T-IF can also be designed to provide the trained function TF.

The training device TRS can also be designed to perform a proposed computer-implemented method for providing a trained function TF. The training device TRS can be designed in particular to perform the embodiment variants of the method for providing a trained function TF by virtue of the training interface T-ST and the training computing unit T-CU being designed to perform the respective steps of the method.

The processing unit 45 and/or the training device TRS may be in particular a computer, a microcontroller or an integrated circuit. Alternatively, the processing unit 45 and/or the training device TRS may be a real or virtual interconnection of computers (a real interconnection is referred to as a “cluster” and a virtual interconnection is referred to as a “Cloud”). The processing unit 45 and/or the training device TRS may also be embodied as a virtual system, which is implemented on a real computer or a real or virtual interconnection of computers (virtualization).

An interface IF and/or a training interface T-IF may be a hardware or software interface (for instance PCI bus, USB or FireWire). A computing unit CU and/or a training computing unit T-CU may comprise hardware elements or software elements, for instance a microprocessor or what is known as a field programmable gate array (FPGA). A memory unit MU and/or a training memory unit T-MU may be implemented as a non-permanent main memory (random access memory or RAM for short) or as a permanent mass storage device (hard disk, USB stick, SD card, solid state disk).

The interface IF and/or the training interface T-IF may comprise in particular a plurality of sub-interfaces, which implement different steps of the respective methods. In other words, the interface IF and/or the training interface T-IF can also be regarded as a multiplicity of interfaces IF or a multiplicity of training interfaces T-IF respectively. The computing unit CU and/or the training computing unit T-CU may comprise in particular a plurality of sub-computing units, which implement different steps of the respective methods. In other words, the computing unit CU and/or the training computing unit T-CU can also be regarded as a multiplicity of computing units CU or a multiplicity of training computing units T-CU respectively.

In the example embodiment shown, the processing unit 45 is connected to the training device TRS via a network NETW. In addition, the processing unit 45 is connected directly to a control unit 51 that is designed to control a medical apparatus 32 coupled thereto. In particular, the medical apparatus can comprise the control unit 51. The connection to the control unit 51, or to the medical apparatus 32, can also be made via the network NETW, however. The medical apparatus 32 can also comprise the processing unit 45.

In addition, the communication between the processing unit 45 and the training device TRS can also take place offline, for instance by exchanging data storage media. For example, communication between the processing unit 45 and the training device TRS can consist in the processing unit 45 transferring additional training function-datasets and comparison function-datasets to the training device TRS, or in the training device TRS transferring the trained function to the processing unit 45. In addition, the training device TRS can also be connected to other data sources.

The network NETW may be a local area network (LAN) or a wide area network (WAN). An example of a local area network is an intranet; an example of a wide area network is the Internet. The network NETW can be implemented in particular also wirelessly, in particular as WLAN (wireless LAN, commonly known as WiFi) or as a Bluetooth connection. The network NETW can also be implemented as a combination of the examples mentioned.

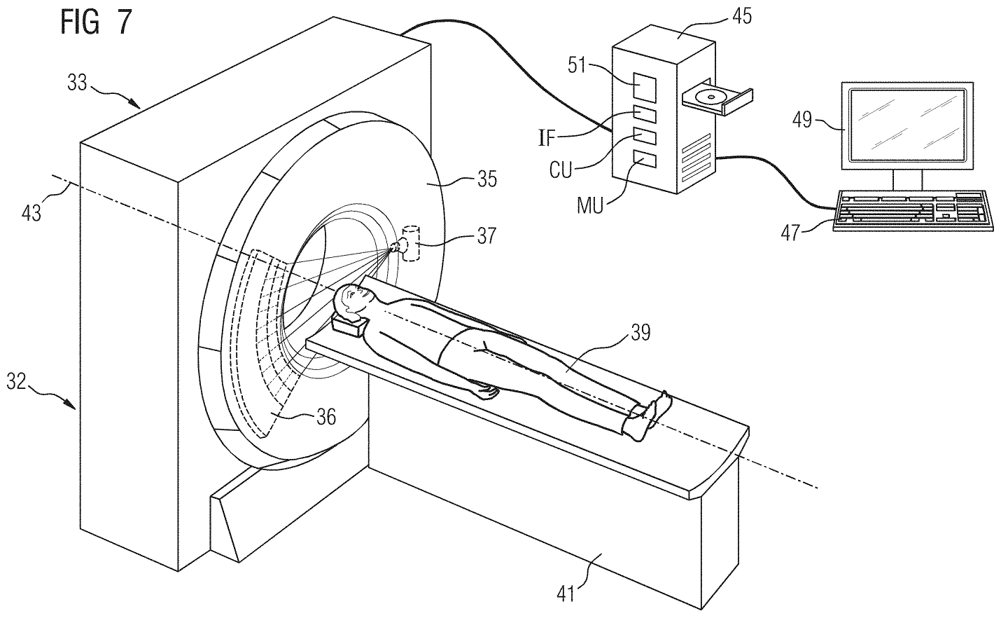

FIG. 7 shows an example embodiment of a medical apparatus 32 according to the invention. In particular, the medical apparatus 32 is a medical imaging apparatus. In the example shown, the medical imaging apparatus is in the form of a computed tomography apparatus.

The computed tomography apparatus comprises a gantry 33 having a rotor 35. The rotor 35 comprises a radiation source or X-ray source 37, in particular an X-ray tube, and an X-ray detector unit 36 opposite thereto. In the case of the CT apparatus, the X-ray detector unit 36 and the X-ray source 37 can also be referred to jointly as a data acquisition unit. The X-ray detector unit 36 and the X-ray source 37 can rotate about a common axis 43 (also called the axis of rotation z). The patient 39 is supported on the patient positioning device 41 and can be moved along the axis of rotation z 43 by the gantry 33. In general, the patient 39 can include, for example, an animal patient and/or a human patient.

In the case of a computed tomography apparatus, the X-ray detector unit 2 typically is used to acquire a (raw) X-ray image dataset of the patient 32 from a multiplicity of angular directions while the patient positioning device 41 moves the patient continuously or sequentially through the gantry 33. Then a mathematical method, for instance comprising filtered backprojection or an iterative reconstruction technique, can be used to reconstruct a final X-ray image dataset on the basis of the (raw) X-ray image dataset.

The medical apparatus comprises a processing unit 45.

The processing unit 45 comprises a control unit 51 of the medical apparatus. In addition, the processing unit 45 comprises a computing unit CU, an interface IF and a memory MU. The processing unit 45 and the control unit 51 can advantageously be designed to perform a method according to the invention for controlling a medical apparatus, in this case the CT apparatus.

In addition, an input device 47 and an output device 49 are connected to the processing unit 45. The input device 47 and the output device 49 can facilitate, for example, an interaction, for instance a manual configuration, an actuation or an initiation of a method step by a user.

A simple example of a specific case in which a method for controlling a medical apparatus can be used for improved control is the control of the CT apparatus on the basis of an EKG dataset of the patient.

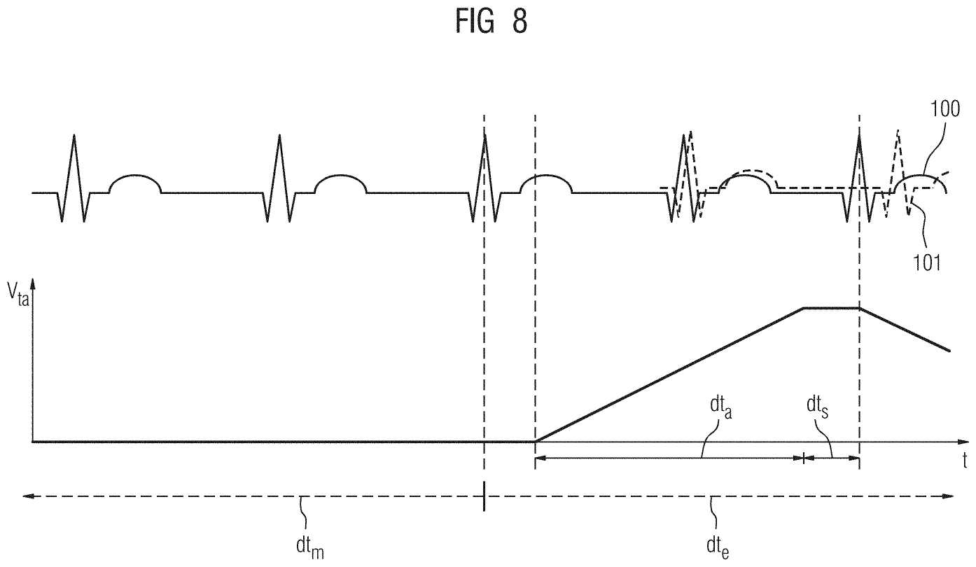

For the situation in which, for instance, a data acquisition sequence using the CT apparatus is planned in a time interval between 60% and 80% of a cardiac cycle, i.e. starting at 60% of the timespan between two R-spikes of the EKG, it is necessary for control of the patient positioning device 41 to take place promptly already in the lead-up thereto so that the patient 39 is optimally positioned and the speed of the patient positioning device 41 during the scan phase is matched to the data acquisition sequence. FIG. 8 shows in a sketch the timing in such a situation by way of example. A patient positioning device 41 is accelerated during an acceleration phase dtₐ to a constant speed vₜₐ during the planned scan phase dtₛ. In order to facilitate this, control of the patient positioning device 41 must already be performed in a preceding cardiac cycle. Hence a prediction of the cardiac cycles or at least the time length of the respective cardiac cycles is needed for sufficiently early and correct control of the patient positioning device 41 and/or also of the data acquisition device 36,37.

For an estimate of the cardiac cycles in a second time interval dtₑ on the basis of measured cardiac cycles in a first time interval dtm, an inaccurate estimate 101 that deviates from an actual course 100 of the EKG dataset, and control of the medical apparatus 32 based on such an inaccurate estimate 101, can lead to a significant deterioration in the results, in this case in the image quality, because it is not possible to ensure well-timed or optimum control. In this case, control based on the inaccurate prediction would take place too late.

In addition, as accurate an estimate as possible can be required when a speed of a patient positioning device 41 is being controlled in a manner matched to the cardiac rate during table incrementation that is continuous throughout the data acquisition, in order that the patient 39 is positioned relative to the data acquisition unit 36,37 in successive cardiac cycles optimally for the data acquisition.

Another usage can include using a medical X-ray apparatus, for instance a CT apparatus 32, to acquire image data, where the aim is to acquire data during the same specified cardiac phase in each successive cardiac cycle while simultaneously minimizing an X-ray dose administered to a patient 39. Systematic control of the medical apparatus, in particular of the radiation status of the radiation source, in order for X-ray radiation to be emitted solely in the specified phases of the successive cardiac cycles, for instance between 60% and 80% of each cardiac cycle, or at least to reduce the emitted radiation dose in intervening phases, while having to ensure full data acquisition at the same time, requires as accurate an estimate as possible for the control. The method according to the invention can advantageously ensure an improved estimate.

In particular, an additional external influencing event IE, for instance a breath signal for the patient 39 to hold their breath during a data acquisition, or the injection of a contrast agent, can also here influence the patient parameters used for the control and thereby create problematic conditions for an estimate, and/or can necessitate a waiting period until the patient parameters have stabilized. The method according to the invention can facilitate an improved estimate in this case. Equally, waiting periods can be avoided, because it is also possible to take better account of such influencing events IE.

It is also easy to derive similar scenarios and cases of use on the basis of other patient parameters, for instance control based on the patient's respiration, and also for other medical apparatuses.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for controlling a medical apparatus, comprising:
providing, via an interface, a first function dataset of a patient, measured within a first time interval;
applying, via a processor, a trained function to the measured first function dataset provided, to estimate a second function dataset of the patient, predicted for a second time interval, wherein at least one parameter of the trained function is adapted based upon a comparison between a predicted, second training function-dataset for a second training time-interval, the second training function-dataset being predicted based upon a first training function-dataset of a training patient for a first training time-interval, and a comparison function-dataset of the training patient for the second training time-interval, and wherein the first training function-dataset and the comparison function-dataset are associated; and
controlling, via a controller, the medical apparatus based upon the estimate.

2. The method of claim 1, wherein the first time interval and the measured, first function dataset are updated, and wherein a predicted, second function dataset is re-estimated for an updated, second time interval by applying the trained function to the measured, first function dataset after being updated.

3. The method of claim 1, wherein the measured, first function dataset is based on at least one dataset from one of:
an EKG dataset of the patient;
a respiratory dataset of the patient;
a blood-pressure dataset of the patient; or
a blood-level dataset of the patient.

4. The method of claim 1, wherein the measured, first function dataset of the patient) is influenced at least by an influencing event occurring during the first time interval, and wherein the trained function is adapted based upon a first training function-dataset of a training patient, the training function-dataset being influenced by a training influencing event.

5. The method of claim 4, wherein the influencing event comprises at least one of:
a breath signal defined for the patient;
a medication of the patient; or
a contrast-agent injection.

6. The method of claim 1, wherein the second time interval is immediately adjacent in time to the first time interval.

7. The method of claim 1, wherein the controlling comprises:
activating the medical apparatus or a component of the medical apparatus;
controlling a movement of the medical apparatus or a component of the medical apparatus;
starting or stopping a data acquisition sequence via a data acquisition device of the medical apparatus; or
adjusting a radiation status of an X-ray source of the medical apparatus.

8. The method of claim 1, wherein the trained function comprises a neural network.

9. A training method for providing a trained function, comprising:
providing, via a training interface, a first training function-dataset of a training patient for a first training time-interval and an associated comparison function-dataset of the training patient for a second training time-interval;
applying, via a training computer, the trained function to the first training function-dataset provided to estimate a predicted, second training function-dataset of the training patient in the second training time-interval;
adapting, via the training computer, at least one parameter of the trained function based upon a comparison of the predicted, second training function-dataset and a corresponding comparison function-dataset in the second training time-interval; and
providing, via the training interface, the trained function.

10. A device for controlling a medical apparatus, comprising:
a processor including a computing device;
an interface designed to provide a first function dataset of a patient, measured within a first time interval; and
a computing unit, designed to apply a trained function to the first function dataset measured, for estimating a predicted, second function dataset of the patient in a second time interval, at least one parameter of the trained function being adapted based upon a comparison of a second training function-dataset for a second training time interval, the second training function-dataset being predicted based upon a first training function-dataset of a training patient for a first training time-interval, and a corresponding comparison function-dataset of the training patient for the second training time-interval; and
a controller, is designed to control the medical apparatus based upon the predicted, second function dataset of the patient, once estimated.

11. A medical apparatus comprising the device of claim 10.

12. A training device for providing a trained function, comprising:
a training interface designed to provide a first training function-dataset of a training patient for a first training time-interval and an associated comparison function-dataset of the training patient for a second training time-interval; and
a training computing unit
designed to apply a trained function to the first training function-dataset to estimate a predicted, second training function-dataset of the training patient in the second training time-interval,
designed to adapt at least one parameter of the trained function based upon a comparison of the predicted, second training function-dataset and the corresponding comparison function-dataset in the second training time-interval, and
designed to provide the trained function.

13. A non-transitory computer program product storing a computer program, directly loadable into a memory of a processor, including program segments to perform the method of claim 1 when the program segments are executed by the processor.

14. A non-transitory computer-readable storage medium, storing program segments, readable and executable by a processor, to perform the method of claim 1 when the program segments are executed by the processor.

15. The method of claim 2, wherein the measured, first function dataset is based on at least one dataset from on of:
an EKG dataset of the patient;
a respiratory dataset of the patient;
a blood-pressure dataset of the patient; or
a blood-level dataset of the patient.

16. The method of claim 2, wherein the measured, first function dataset of the patient) is influenced at least by an influencing event occurring during the first time interval, and wherein the trained function is adapted based upon a first training function-dataset of a training patient, the training function-dataset being influenced by a training influencing event.

17. The method of claim 16, wherein the influencing event comprises at least one of:
a breath signal defined for the patient;
a medication of the patient; or
a contrast-agent injection.

18. The method of claim 2, wherein the second time interval is immediately adjacent in time to the first time interval.

19. The method of claim 2, wherein the controlling comprises:
activating the medical apparatus or a component of the medical apparatus;
controlling a movement of the medical apparatus or a component of the medical apparatus;
starting or stopping a data acquisition sequence via a data acquisition device of the medical apparatus; or
adjusting a radiation status of an X-ray source of the medical apparatus.

20. The method of claim 2, wherein the trained function comprises a neural network.

21. A non-transitory computer program product storing a computer program, directly loadable into a training memory of a training device, including program segments to perform the method of claim 9 when the program segments are executed by the training device.

22. A non-transitory computer-readable storage medium, storing program segments, readable and executable by a training device, to perform the method of claim 9 when the program segments are executed by the training device.
